(19) United States
(12) Patent Application Publication
(10) Pub. No.: US 2008/0077455 A1
(43) Pub. Date: Mar. 27, 2008

(54) METHOD AND SYSTEM FOR SELLING FOOD AT RESTAURANT

(76) Inventor: ISRAEL GILBOA, Karmiel (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 11/535,112

(22) Filed: Sep. 26, 2006

(51) Int. Cl. G06Q 10/00

(52) U.S. Cl. 705/5

(57) ABSTRACT

A method for ordering a meal at a restaurant. Nutritional information of the meal is transferred, and the meal is ordered based on the nutritional information such as on the energy content of the meal and a description of the meal. The meal is assembled based on the nutritional information. A purchase price of the meal, is preferably calculated based on the nutritional information, for instance based solely on the energy (caloric) content of the meal. The nutritional information is preferably logged including a time (and day) of consumption of at least part of the meal. A computerized data base is preferably stored in memory of a computer. The data base includes dietary information of at least one food component, and a scale is operatively attached to the computer. A weight is calculated of an ingredient of the meal based on the nutritional information and the dietary information. Using the scale, the ingredient is weighed and adjusted to approach the calculated weight.

### FIELD AND BACKGROUND OF THE INVENTION

The present invention relates to a system and method for ordering meals in a restaurant, particularly in a fast food restaurant. Specifically, the method includes ordering a meal and/or drink in which the quantities of ingredients of the meal and/or portion size are based on nutritional information or requirements transferred when ordering the meal.

Over the past decade, people have been increasingly concerned with nutrition and diet for a number of reasons including general health maintenance, reducing obesity, increasing longevity, body building or just to feel good. Governmental bodies such as United States Department of Agriculture have published nutritional recommendations for maintaining good health. These recommendations include specific daily intake of meats or beans, whole grains, fruits, vegetables and milk products in addition to specific daily caloric intake. Furthermore, dietitians, nutritionists, trainers and doctors may diets, however little if any accurate feedback is available to determine to what extent the prescribed diets are being followed.

While eating at home, people generally have greater control over the ingredients of food and drink they prepare and consumer however, while eating out in cafeterias and restaurants it is difficult to estimate the nutritional content of food and drink ordered, purchased and consumed.

There is considerable prior art in the area of ordering meals from restaurants.

United States patent application publication, 20040069313, discloses a menu which includes information about each food item selection concerning that item's compliance with certain diets and diet type. With this information, a customer may eat out and remain on the dietary or nutritional plan of his/her choice. With the customers' selection of a food item, the kitchen of the restaurant may prepare the food item by selecting ingredients, cooking methods, and side dishes in compliance with the selected diet or nutrition plan.

United States patent application publication 20050075934 discloses a consumer user interface for a point of sale food selection system which is modified based on certain dietary requirements. The system and method may be applied to point of sale kiosks as well as wireless data devices for use in restaurants, delis, cafeterias, coffee shops and other food retail environments. During the process of ordering, the interface determines a user's preference for one or more dietary requirements. Once the preferences are determined, subsequent choices for an order may be suggested, highlighted, recommended, parsed, or otherwise modified to aid the consumer in selecting meals that are tailored to their individual preferences.

United States Patent Application 20050171800 discloses a menu-order selection support system for supporting a person in ordering an item by selecting and providing food and drink menus at an eating-house or a restaurant. A user performs health management wherever the user takes a meal, even if the user takes a meal at multiple places. A data center manages personal data including preference information, harmful foodstuff information. Health management information is provided from the data center in response to a request from the restaurant for receiving customer's personal data the customer is provided with a menu that matches the customer's personal data, when the restaurant system is connected to the data center via a communication line.

In order to exemplify the prior art, Sam and Rosie are looking for a place to eat lunch downtime. Rosie although not strictly on any particular diet considers carefully the nutritional content of the food she eats. Sam notices a burger place and suggests they enter. After entering and waiting on a short line, Rosie inquires from the proprietor, “What is the calorie and fat content of the regular burger and bun?”. The proprietor responds, “No idea!”. Rosie indicates to Sam that they should look for another place, despite their impending hunger. Next Rosie and Sam stumble into a full service restaurant which has an option to operate according to the disclosure of US20040069313. After sitting down, Rosie and Sam receive menus. The menu includes a section on diet meals according to well known published diets. Although Rosie isn't on a particular diet, she requested a salad and fish according to a well known diet. Sam then noticed that the price of the diet meals are considerably higher than the regular meals, presumably because they are prepared and cooked specially. A diner sitting at a nearby table hears that Rosie and Sam are discussing the diet meals offered at the restaurant and he said, “I ordered one of those diet meals once and it took forever until it was served. I suggest fried fish and chips”. Rosie not wanting to eat fried fish pulled Sam hungry as he was to search for another place to eat.

There is thus a need for, and it would be highly advantageous to have a method of ordering a meal and/or drink in a restaurant in which the meal is assembled based on nutritional information provided by the client

Reference is made to FIG. 3 (prior art) illustrating a computer 22, which includes a processor 301, a storage mechanism including a memory bus 307 to store information in memory 309 and a network interface 305 operatively connected to processor 201 with a peripheral bus 203. Computer 22 further includes a data input mechanism 311, e.g. disk drive and a program storage device 313, e.g. optical disk. Data input mechanism 311 is connected to processor 301 with peripheral bus 303. A display 26 and input device 315, e.g. mouse, keyboard are connected to processor 301 with peripheral bus 303.

### DEFINITIONS

The terms “nutritional requirement(s)” and “nutritional information are used herein interchangeably. The terms “energy” content” and “caloric content” are used herein interchangeably. The terms “food component” and “ingredient” are used herein interchangeably. The term “time” as used herein, referring to logging, includes time of day and date and/or day of week. The term “meal” is used herein to refer to a single dish or drink of multiple ingredients, or meal including multiple dishes, for instance a main dish and a side dish. The term “assembly” of a meal (or drink) as used herein refers to a simple combination of the ingredients, components or portions of a meal or drink based on weight or volume of the ingredients, components or portions. The term “assembly” of a meal differs from the term “preparation” of a meal. The term “‘preparation” of a meal refers to assembling ingredients in addition to other actions such as mixing, and/or cooking, or baking,

### SUMMARY OF THE INVENTION

According to the present invention there is provided a method for ordering a meal at a restaurant. Nutritional information of the meal is transferred, and the meal is ordered based on the nutritional information such as based on the energy content of the meal and a description of the meal. The meal is drink or includes a drink and the drink is preferably mixed based on the nutritional information of the drink. The meal is assembled based on the nutritional information. A purchase price of the meal, is preferably calculated based on the nutritional information, such as based solely on the energy (caloric) content of the meal. The nutritional information is preferably logged preferably including a time (and day) of consumption at least in part of the meal. A computerized data base is preferably stored in memory of a computer. The data base includes dietary information of at least one food component, and a scale is attached to the computer. A weight is calculated of a food component of the meal based on the nutritional information and the dietary information. Using the scale, the food component of the meal is weighed and adjusted to approach the calculated weight. The nutritional information applies to the food components of the meal such as fats, carbohydrates, sugars, vitamins, minerals, meats, milk products, fish, beans, fruits, vegetables and grains and/or specifies energy (caloric) content of the meal the transferring of nutritional information is preferably performed by transferring, a data storage medium from a client of the restaurant.

According to the present invention there is provided a system for ordering a meal by a client at a restaurant includes the computer, the computerized data base and the scale A transfer mechanism transfers nutritional information of the meal to be assembled. A data base is operatively attached to a computer with dietary information of food components used at the restaurant. A weight is calculated by the computer of an ingredient or a food component of the meal based on the nutritional information and the dietary information. A scale, attached to the computer, weighs the ingredient of the meal, and the ingredient is adjusted for instance by the patron of the restaurant to approach the calculated weight. A cash register is operatively attached to the computer. The cash register registers a cost of the meal based on the nutritional information. The transfer mechanism preferably includes a data storage medium. A logging mechanism logs the nutritional information of the meal and indicates consumption of at least a portion of the meal and a time of said consumption, preferably on the data storage medium.

According to the present invention there is provided a program storage device, readable by a computer, tangibly embodying a program of instructions executable by the computer to perform a method for assembling a meal. A first data base storing recipes is attached to the computer and a second data base attached to the computer, stores dietary information of food components used in the recipes. Nutritional information required of the meal is input with a description of the meal. A recipe is input from the first data base based on the description. For each food component used in the recipe, the quantity of the food component is optimized based on the nutritional information and an instruction is displayed to add the optimized quantity to the meal. Preferably, the computer is attached to a scale, a weight of the food component is input from the scale to the computer. The quantity of consumption of at least part of the meal is logged and the time of the consumption. The log is preferably maintained on the same data storage mechanism used to input the nutritional information.

### BRIEF DESCRIPTION OF THE DRAWINGS

The invention is herein described, by way of example only, with reference to the accompanying drawings, wherein:

FIG. 1 is a simplified system drawing, according to an embodiment of the present invention;

FIGS. 2 and 2a are flow drawings, according to an embodiment of the present invention;

FIG. 3 is a prior art drawing of a computer and peripherals used to implement embodiments of the present invention;

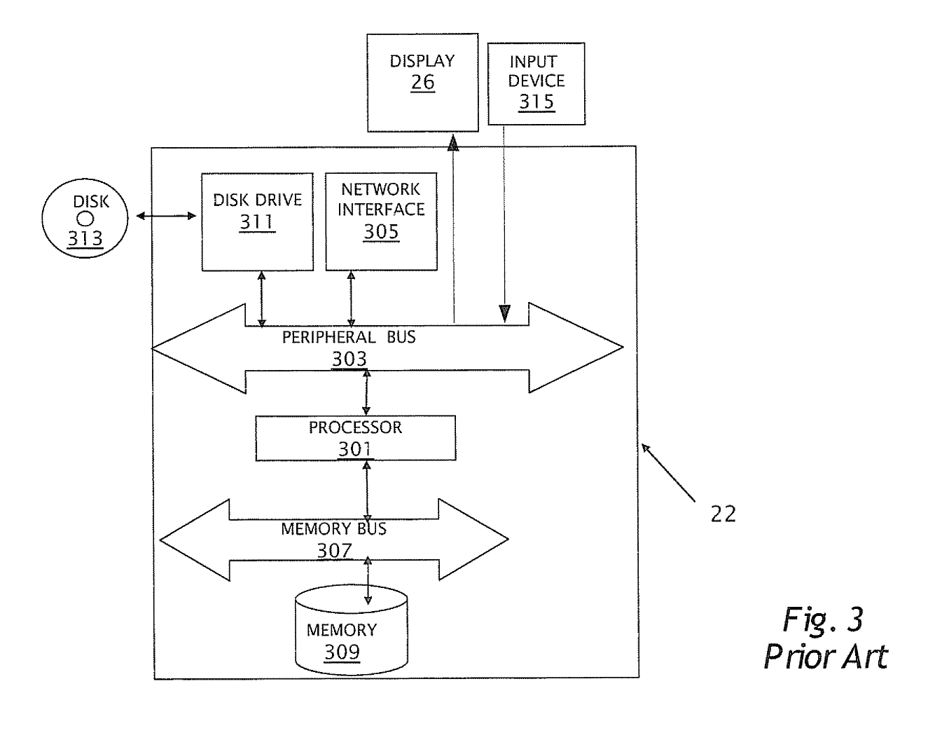

FIG. 4 is a flow chart of an application implemented using the computer of FIG. 3, according to an embodiment of the present invention

### DESCRIPTION OF THE PREFERRED EMBODIMENTS

The present invention is of a system and method of ordering meals in a restaurant, particularly in a fast food restaurant. Specifically, a client of the restaurant provides nutritional information of the meal or drink to be ordered. The method includes ordering a meal and the meal is assembled based on the nutritional information; or a drink is ordered based on the nutritional information, e.g. calorie content, of the drink. The purchase price of the meal is preferably calculated based on the nutritional information.

The principles and operation of a system and method of ordering a meal and/or drink in which the meal is assembled and/or portion size are based on nutritional information of the meal, according to the present invention, may be better understood with reference to the drawings and the accompanying description.

It should be noted, that although the discussion herein relates to fast food restaurants, cafeterias, diners and coffee houses the present invention may by non-limiting example, alternatively be configured as well in conventional full service restaurants for instance at a salad bar. Although the discussion herein relates to use of a scale and meals are assembled according to embodiments of the present invention based on weight, the present invention may be equivalently configured with the meals assembled based on measurement of volume. Measurement of volume is particularly applicable to serving drinks, according to embodiments of the present invention.

According to an embodiment of the present invention, cost of the meal is based solely on nutritional information, e.g caloric content of the meal. Consequently, payment for the meal is optionally arranged prior to preparing the meal based on the nutritional information.

The embodiments of the present invention may include a general-purpose or special-purpose computer or computer system including various computer hardware components. Embodiments within the scope of the present invention also include computer-readable media for carrying or having computer-executable instructions, computer-readable instructions, or data structures stored thereon. Such computer-readable media may be any available media, which is accessible by a general-purpose or special-purpose computer system. By way of example, and not limitation, such computer-readable media can include physical storage media such as RAM, ROM, EPROM, CD-ROM or other optical disk storage, magnetic disk storage or other magnetic storage devices, or any other media which can be used to carry or store desired program code means in the form of computer-executable instructions, computer-readable instructions, or data structures and which may be accessed by a general-purpose or special-purpose computer system.

In this description and in the following claims, a “computer” is defined as one or more software modules, one or more hardware modules, or combinations thereof, which work together to perform operations on electronic data. For example, the definition of computer system includes the hardware components of a personal computer, as well as software modules, such as the operating system of the personally computer. The physical layout of the modules is not important. A computer system may include one or more computers coupled via a computer network. Likewise, a computer system may include a single physical device (such as a mobile phone or Personal Digital Assistant “PDA”) where internal modules (such as a memory and processor) work together to perform operations on electronic data.

Those skilled in the art will appreciate that the invention may be practiced in network computing environments with many types of computer system configurations, including mobile telephones, PDA's, pagers, hand-held devices, laptop computers, personal computers, multi-processor systems, microprocessor-based or programmable consumer electronics, network PCs, minicomputers, mainframe computers, and the like. The invention may also be practiced in distributed computing environments where local and remote computer systems, which are linked (either by hardwired links, wireless links, or by a combination of hardwired or wireless links) through a communication network, both perform tasks. In a distributed computing environment, program modules may be located in both local and remote memory storage devices.

Before explaining embodiments of the invention in detail, it is to be understood that the invention is not limited in its application to the details of design and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

By way of introduction, a principal intention of the present invention is to facilitate people to maintain good health by ordering food, especially fast food, such as pizza, burgers, sandwiches, salads and soft drinks according to nutritional content rather by serving size and description. Another intention of the present invention is to provide a mechanism especially when eating out to maintain an accurate record or log of the nutritional content, ego calories, consumed. The record may be maintained over time and used by the people to maintain their diets and/or by professional clinicians in their service such as dietitians, nutritionists, physicians and trainers. Another intention, according to embodiments of the present invention is to price the meal according to nutritional content, e.g. energy content or calories. Since a high caloric meal costs more than a low caloric meal, the client is provided a monetary inventive to maintain his/her diet. Embodiments of the present invention may be applied in various situations. A parent may wish to monitor the nutritional content of meals purchased by a child at school. Nutritional requirements are transferred to the school, meals are provided to the child according to an embodiment of the present invention and the nutritional content of the meals that the child purchases and consumes is monitored by the parent. In another application, an employee who takes meals at his workplace may agree to have his meals provided according to embodiments of the present invention so that his health insurance is subsidized. In another application, a trainer of a sports team may require players to purchase meals according to an embodiment of the present invention, allowing the trainer to monitor the nutritional value of the food his players are consuming. The monitoring may be performed on local storage, or remotely over a network connection. Another intention of the present invention is provide a computer assisted method to assemble meals for people with allergies and/or other food sensitivities, e.g. gluten, sensitivity and/or limit inadvertent consumption of specific food components, e.g. cholesterol.

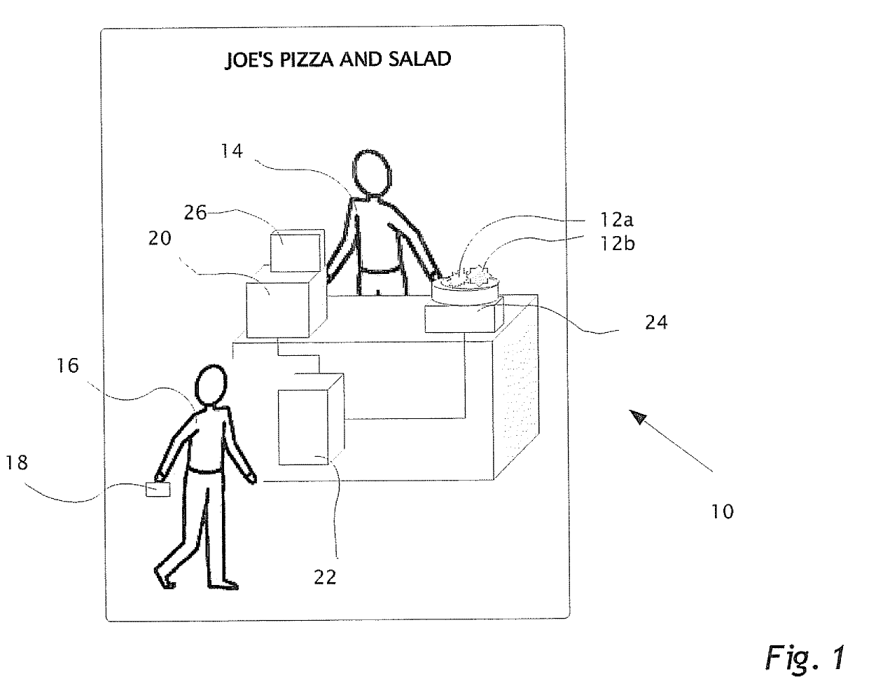

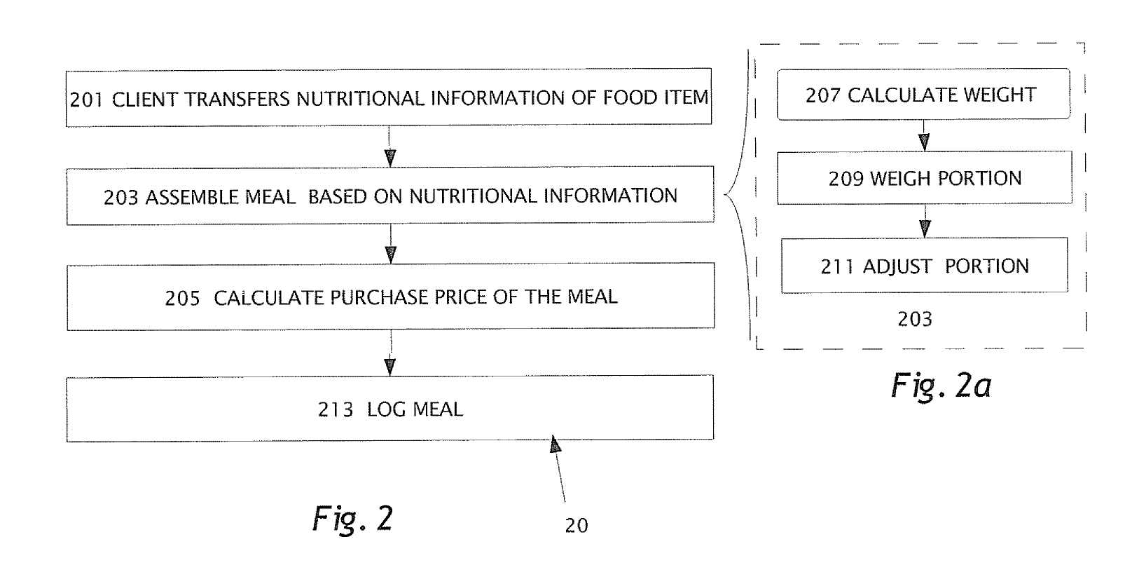

Referring now to the drawings, FIG. 1 illustrates a fast food restaurant 10, according to an embodiment of the present invention. A patron 14 of the restaurant is serving client 16. Restaurant 10 is equipped with a system, according to an embodiment of the present invention including a computer 22, attached to a display 26, to one or more input devices 315 (not shown in FIG. 1) and optionally to a scale 24 which is operable to weigh portions or individual ingredients of food served at restaurant 10. Computer 22 is optionally connected to cash register 20. Different portions of food 12a and 12b are shown on scale 24. Reference is now also made to FIGS. 2 and 2a, a flow diagram of a method, according to an embodiment of the present invention. Returning now to Rosie and Sam, who (now very hungry) approach restaurant 10 Joe's Pizza and Salad, equipped according to an embodiment of the present invention. Sam and Rosie notice on display several “standard” salads and pizza varieties with markings in calories, e.g 450 calories optionally with other nutritional information. Restaurant 10 also includes a data terminal (not shown) useful for clients to retrieve nutritional information of any of the standard meals. Rosie (client 16) wishing to order a “custom meal” transfers (step 201) nutritional information regarding a meal she wishes to order. Nutritional information may be verbally transferred, for instance “A Tuna Nicoise salad of two hundred fifty calories”. Alternatively, Rosie (client 16) transfers more detailed nutritional information for instance in writing or by using a data storage medium 18 (in FIG. 1) such as a magnetic card 18. A sample data structure including nutritional information of the nicoise salad that Rosie wishes to order is found in Table 1.

TABLE 1
Nutritional Information
TUNA NICOISE SALAD

| | | |
|---|---|---|
| Serving size | 300 | g |
| Calories | 250 | |
| Calories from Fat | 100 | |
| Total Fat | 13 | g |
| Saturated Fat | 1.5 | g |
| Trans Fat | 0 | g |
| Cholesterol | 240 | mg |
| Sodium | 880 | g |
| Total Carbohydrates | 15 | g |
| Dietary Fiber | 4 | g |
| Sugar | 3 | g |
| Protein | 25 | g |
| Vitamin A | | |
| Vitamin C | | |
| Calcium | | |
| Iron | | |

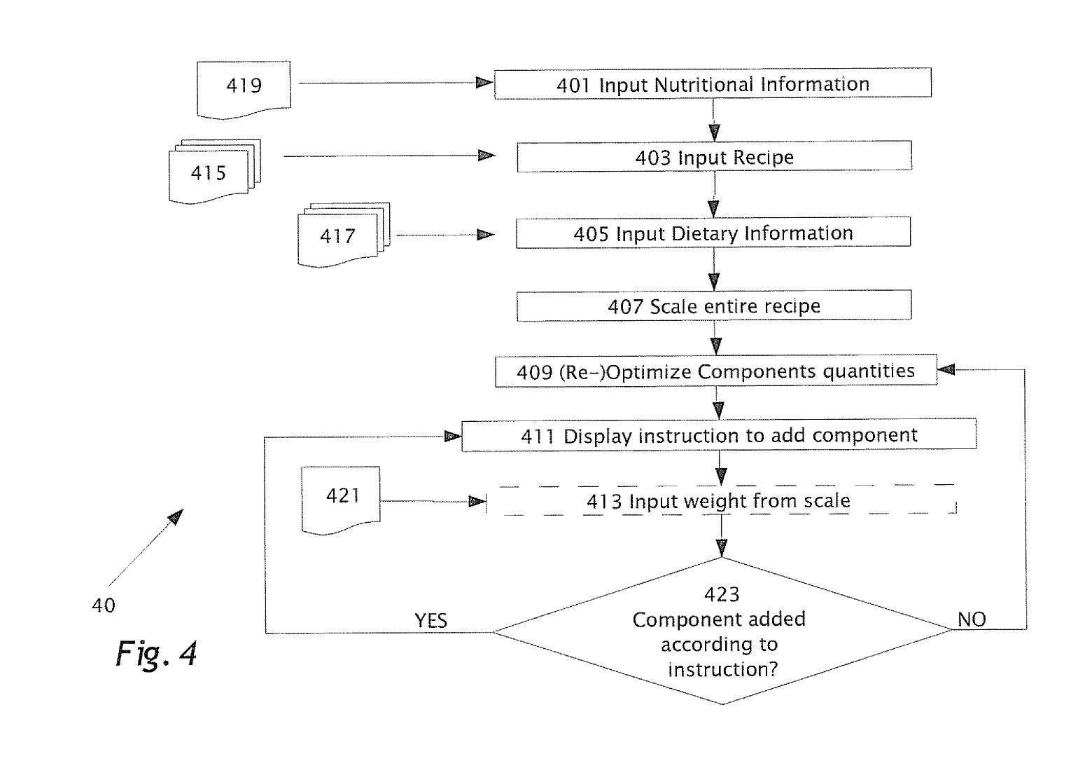

Upon transferring (step 201) nutritional information and reading the nutritional information and optionally storing in memory 309 of computer 22, then Joe (patron 14) assembles (step 203) the Nicoise salad based on the nutritional information. Reference is now made to FIG. 4 which illustrates a software application 40 as previously installed configured and running on computer 22, according to an embodiment of the present invention. Software application 40 inputs (step 401) one or more nutritional requirements 419 (as in Table 1). Software application inputs (step 403) a recipe, e.g. Nicoise salad from a data base 415. of recipes Software application 40 is preferably connected to a second data base 417 including dietary information of ingredients, used to prepare recipes 415. Application 40 locates a basic recipe for Nicoise salad and inputs (step 405) dietary information of the ingredients required, e.g. tuna, olives, anchovies, olive oil for Nicoise salad) and preferably scales (step 407) the entire recipe to the weight in grams as requested in nutritional requirement 419. Application 40 then optimizes (step 409) weights (or volumes) of different ingredients (or components) of the salad in order to comply closely with nutritional requirement 419 for instance as specified by Rosie (client 16) in restaurant 10. In step 411, an instruction is preferably displayed for instance to Joe (patron 14 of restaurant 10). For instance, an instruction to add three anchovies, is displayed (step 411) and Joe optionally responds that the instruction was completed by inputting (not shown) on input device 315. Alternatively, or in addition, application 40 receives confirmation when weight 421 of the salad being assembled (step 203) is updated (step 413) from scale 24. In any case, if the anchovies for instance were added according to instruction in decision block 423 then a new instruction is displayed (step 411), for instance to add olive oil. Otherwise, an instruction was not performed correctly, 40 application may be directed to re-optimize (step 409) the quantities of one or more of the ingredients not as yet assembled.

Referring back to FIGS. 2 and 2a, when Joe (patron 14) views an instruction on display 26, he either adds the ingredient directly to the plate on scale 24 or measures volume first as required aid then places on the ingredient on the plate. The ingredient is weighed (step 209). Optionally, if the weight is too much, Joe optionally adjusts (step 211) the ingredient, e.g. removes an anchovy from the plate or requests computer application 40 to re-optimize (step 409).

When Joe completed assembling (step 203) Rosie's salad. Joe turns to Sam, and asks, “ . . . and for you?”. Sam points at a regular slice of pizza and Joe handed to Sam his slice pizza. Sam asks how much does he owe for the meals. Joe responds, “Your pizza costs $5.00 and the lady's salad costs $2.50. Sam appeals surprised that the small slice of pizza costs more than the large salad. Joe responds, “In this place, you pay a cent per calorie, the slice of pizza has five hundred calories and the salad two hundred and fifty calories. After paying and receiving a receipt preferably including a detailed nutritional content of both meals as ordered, Sam and Rosie finally sit down to eat.

Sam devoured his pizza rather quickly and still looked rather hungry. Rosie took her are time with the salad and when she was satiated, she let Sam finish her salad. Sam thanks Rosie and says, “Next time we are here, I will order a salad”. Joe approaches the couple and asked, “How about a drink” We have orange juice for 50, 100 and 150 calories. Rosie order a 50 calorie drink or fifty cents (at 1 cent per calorie) and for free diluted her drink with soda water at a free soda water fountain, since soda water has negligible calories. Joe asks, “Would you like to log (step 213) your meal?” Rosie hands to Joe magnetic card 18 which she used to transfer nutritional information and said, “Only half the salad, Sam ate the other half.

While the invention has been described with respect to a limited number of embodiments, it will be appreciated that many variations, modifications and other applications of the invention may be made.

What is claimed is:

1. A method for ordering a meal at a restaurant, the method comprising the steps of:
(a) transferring nutritional information of the meal;
(b) ordering the meal, wherein said ordering is based on the nutritional information of the meal and a description of the meal; and
(c) assembling the meal based on the nutritional information.

2. The method, according to claim 1, wherein said ordering is based on the energy content of the meal.

3. The method, according to claim 1, wherein said meal includes a drink and wherein said assembling the meal includes mixing said drink based on the nutritional information of the drink.

4. The method, according to claim 1, further comprising the step of:
(d) calculating a purchase price of the meal, wherein the purchase price of the meal is based on said nutritional information.

5. The method, according to claim 1, wherein said purchase price is based solely on the energy content of the meal.

6. The method, according to claim 1, further comprising the step of:
(d) logging at least in part said nutritional information and time of consumption of at least a portion of the meal.

7. The method, according to claim 1, wherein a computerized data base is stored in memory of a computer, the data base including dietary information of at least one food component used in the meal, wherein a scale is operatively attached to the computer, wherein said assembling the meal includes:
(i) calculating a weight of at least one portion of the meal based on said nutritional information and said dietary information;
(ii) using said scale, weighing said at least one portion; and
(iii) adjusting said at least one portion to approach said weight.

8. The method, according to claim 1, wherein said nutritional information applies to least one food component of the meal, wherein said at least one food component is selected from the group consisting of: proteins, fats, carbohydrates, sugars, vitamins, minerals, meats, milk products, fish, beans, fruits, vegetables and grains.

9. The method, according to claim 1, wherein said nutritional information specifies energy content of the meal.

10. The method, according to claim 1, wherein said transferring nutritional information is performed by transferring a data storage medium from a client of the restaurant.

11. A system for ordering a meal at a restaurant, the system including the computer, the computerized data base and the scale of claim 7, the system operable for performing the method steps of claim 7.

12. A system for assembling a meal, the system comprising:
(a) a transfer mechanism which transfers nutritional information as required of the meal;
(b) a data base with dietary information of a plurality of food components wherein said data base is operatively attached to a computer; and
(c) calculating in said computer a weight of at least one of said food components used in the meal, wherein said weight is based on said nutritional information and said dietary information.

13. The system, according to claim 12, further comprising the step of
(d) a scale which weighs said at least one food component, wherein said scale is operatively attached to said computer; wherein said at least one food component is adjusted to approach said weight.

14. The system, according to claim 13, further comprising:
(e) a cash register operatively attached to said computer, wherein said cash register registers a cost of the meal based on said nutritional information.

15. The system, according to claim 12, wherein said transfer mechanism includes a data storage medium.

16. The system, according to claim 12, wherein said transfer mechanism includes a data storage medium, further comprising the step of:
(e) a logging mechanism which indicates consumption of at least a portion of said meal and a time of said consumption.

17. The system, according to claim 16, wherein said logging mechanism is stored by said computer on said data storage medium.

18. A program storage device readable by a computer, tangibly embodying a program of instructions executable by the computer to perform a method for assembling a meal, wherein a first data base storing a plurality of recipes is operatively attached to the computer and a second data base, operatively attached to the computer, stores dietary information of a plurality of food components used in the recipes, the method comprising the steps of:
(a) inputting the nutritional information required of the meal and description of the meal;
(b) from the first data base, inputting a recipe based on said description;
(c) for at least one of the food components used in said recipe, optimizing a quantity of said at least one food component based on said nutritional information; and
(d) displaying an instruction to add said quantity to the meal.

19. The program storage device, according to claim 18, wherein the computer is attached to a scale, wherein the method further comprises the step of:
(e) inputting a weight from the scale of said at least one food component.

20. The program storage device, according to claim 18, wherein the method further comprises the step of:
(e) logging a quantity of consumption of at least a portion of said meal and a time of said consumption.

21. The program storage device, according to claim 20, wherein said (a) inputting said nutritional information is performed using a data storage mechanism and said logging is performed by storing said consumption and time on said data storage mechanism.
